(19) United States
(12) Patent Application Publication
(10) Pub. No.: US 2004/0265387 A1
(43) Pub. Date: Dec. 30, 2004

(54) Super-absorbing hydrogel with specific particle size distribution

(76) Inventors: Dieter Hermeling, Bohl-Iggelheim (DE); Uwe Stuven, Bad Soden (DE); Ulrike Hoss, Kriftel (DE)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/486,808

(22) PCT Filed: Sep. 3, 2002
(86) PCT No.: PCT/EP02/09812

Related U.S. Application Data
(60) Provisional application No. 60/318,337, filed on Sep. 12, 2001.

(30) Foreign Application Priority Data
Sep. 7, 2001 (DE) 101 44 072.3
Jan. 24, 2002 (DE) 102 02 839.7

(51) Int. Cl. A61K 9/14; C08G 63/48; C08G 63/91

(52) U.S. Cl. 424/486; 525/54.2

(57) ABSTRACT

The invention relates to novel hydrophilic swellable polymers with a specific particle size distribution, to the production of the same and to the use thereof for absorbing aqueous liquids, for example in the foodstuff industry, medical field, building and design industries, agricultural industry or fireproofing applications.

The present invention relates to novel hydrophilic swellable addition polymers of a certain particle size distribution, their preparation and their use for absorbing aqueous fluids, for example in the food sector, in medicine, in building construction, in the agricultural industry or in fire protection.

More particularly, the present invention relates to novel hydrophilic swellable acidic and/or postcrosslinked polymers having a particle size distribution of less than 250 μm.

Swellable hydrogel-forming polymers, known as superabsorbent polymers or SAPs, are referred to herein also as hydrogel-forming polymers capable of absorbing aqueous fluids, and are known in principle from the prior art. They are networks of flexible hydrophilic addition polymers, which can be not only ionic but also nonionic in nature. They can optionally be surface postcrosslinked. They are capable of absorbing and binding aqueous fluids by forming a hydrogel and therefore are preferentially used for manufacturing tampons, diapers, sanitary napkins and other hygiene articles in the absorption of body fluids. Within hygiene articles, SAPs are generally accommodated in an absorbent core which, as well as SAP, comprises other materials, including fibers (cellulose fibers), which act as a kind of liquid buffer to intermediately store the spontaneously applied liquid insults and are intended to ensure efficient disbursement of body fluids in the absorbent core and transmission to the SAP.

Hydrogel-forming polymers are in particular copolymers of (co)polymerized hydrophilic monomers, graft (co)polymers of one or more hydrophilic monomers on a suitable grafting base, crosslinked cellulose or starch ethers, crosslinked carboxymethylcellulose, partially crosslinked polyalkylene oxide or natural products that swell in aqueous fluids, for example guar derivatives, alginates and carrageenans.

Suitable grafting bases can be of natural or synthetic origin. Examples are starch, cellulose or cellulose derivatives and also other polysaccharides and oligosaccharides, polyvinyl alcohol, polyalkylene oxides, especially polyethylene oxides and polypropylene oxides, polyamines, polyamides and also hydrophilic polyesters.

Preferred hydrogel-forming polymers are polymers with a high degree of crosslinking and/or surface-postcrosslinked polymers having acid groups, which are predominantly in the form of their salts, generally alkali metal or ammonium salts. Furthermore the preferred acidic hydrogel-forming polymers are those which can be optionally surface postcrosslinked. Such polymers swell particularly strongly and quickly on contact with aqueous fluids to form gels.

Preference is given to polymers which are obtained by crosslinking polymerization or copolymerization of acid-functional monoethylenically unsaturated monomers or salts thereof. It is further possible to copolymerize these monomers without crosslinker and to crosslink them subsequently.

Synthetic products of this type can be prepared by known polymerization processes from suitable hydrophilic monomers, for example acrylic acid. Preference is given to a polymerization in aqueous solution by the process of gel polymerization. It gives rise to polymers in the form of aqueous jellies which are obtained in solid form by known drying processes following mechanical comminution in suitable apparatus.

Water-insoluble yet water-swellable hydrogels are accordingly obtained by incorporation of crosslinking sites in the polymer. It has been determined that the degree of crosslinking is responsible not just for the water solubility of these products but also for their absorption capacity and gel strength. Accordingly, the first generation hydrogels were optimized especially in the direction of high absorption capacities in order that large amounts of cellulose fluff may be saved in the hygiene sector in particular. The trend toward using higher amounts of hydrogel particles and to pack them ever tighter foregrounded other requirements of the absorption profile, such as gel strength or Absorbency Under Load.

As before, especially the use of relatively large amounts of highly swellable hydrogels give rise to the phenomenon of gel-blocking. Gel-blocking occurs when fluid wets the surface of the highly absorbent hydrogel particles and the outer shell swells. The result is the formation of a barrier layer which slows diffusion of liquids into the particle interior. The diffusion times are too short to ensure quantitative absorption. It is thus absolutely necessary, in the hygiene sector for example, for the highly absorbent hydrogel particles to be embedded in an adequate amount of a fiber matrix, which continues to perform the function of fluid distribution and transmission.

Gel-blocking control decisively requires permeability or transportation properties on the part of the tightly packed hydrogels especially at higher use levels (important for use in the agricultural sector). The hydrogel's ability to transmit and distribute fluid is decisive for the channeling of the aqueous fluid to be absorbed not only to neighboring hydrogel particles but also into the particle interior to fully exploit the absorption capacity available. The polymer in the swollen state must not form a barrier layer to subsequent fluid (gel-blocking), as is the case on repeated application of aqueous fluids. The most important criterion is accordingly the ability to transmit fluid in the swollen state. Only this criterion would ensure full exploitation of the actual advantages of hydrogels, namely their pronounced absorption and retention capacity for aqueous fluids.

However, these criteria are only important for certain applications, especially in the hygiene sector. In other applications it can be perfectly desirable for blocking to occur, for example with regard to the use in cable sheathing or in the building industry, where specifically the sealing performance characteristics constitute a significant factor for the assessment of superabsorbent quality.

Another important requirement is a sufficiently fast swell rate for the hydrogel, regardless of the particular application of the highly swellable hydrogel material. Hydrogel swell rate is quantified in the laboratory by measuring the time-dependent AUL with a low pressure (0.014 psi in the experiments) by the Vortex Time test. A defined amount of hydrogel is sprinkled into an aqueous salt solution with stirring and the time is measured in seconds until the vortex in the liquid due to the stirring has closed up and a smooth surface has formed. A Vortex Time test is accordingly a direct measure of the rate of absorption.

There has been no shortage of attempts to avoid gel-blocking and to improve the permeability, although they usually involve an aftertreatment of the particle surface of the hydrogel material.

DE-A-3 523 617 (Nippon Shokubai) and U.S. Pat. No. 4,734,478 (Nippon Shokubai) describe the addition of finely divided amorphous silicas to dry hydrogel powder following surface postcrosslinking with carboxyl-reactive crosslinker substances. U.S. Pat. No. 4,286,082 (Nippon Shokubai) describes mixtures of silica with absorbent but not surface-postcrosslinked-polymers for use in hygiene articles. The purpose of the subsequent addition is to improve the anticaking tendency in moist air and to improve product handling. The finely divided silica is added with an average particle diameter of not more than 10 μm.

EP-A-0 341 951, U.S. Pat. No. 4,990,338 and U.S. Pat. No. 5,035,892 describe the use of silica in the production of antimicrobial absorbent polymers. U.S. Pat. No. 4,535,098 and EP-A-0 227 666 describe the use of colloidal carrier substances based on silica to increase the gel strength of absorbent polymers. EP-A-0 227 666 describes the use of water-insoluble inert inorganic materials (precipitated silica, pyrogens, compounds of aluminum, titanium, zinc, zirconium, nickel, iron-or cobalt) having a preferred primary particle size of 8 to 10 nm. EP 224 923 (Sumitomo) describes the agglomeration of SAP particles by addition of water, silica, surfactant and organic solvent followed by a distillation of the solvent.

WO 95/11932 (Allied Colloids) describes the addition of finely divided silica and/or aluminum salts to the surface postcrosslinker solution to maximize the absorption under high loads.

U.S. Pat. No. 5,314,420 and U.S. Pat. No. 5,399,591 (Nalco) mention the use of polyvalent metal ions as surface postcrosslinkers. U.S. Pat. No. 5,122,544 (Nalco) describes agglomerating superabsorbents with bifunctional epoxides.

EP 386 897 (Nippon Shokubai) describes superabsorbent polymers having a low anticaking tendency and a lower residual monomer content through mixing the polymer granules with aqueous salt solutions, preferably with a combination of Al₂(SO₄)₃ and NaHSO₃. The starting polymers used here have not been subjected to any surface postcrosslinking.

WO 95/26209 (P&G) utilizes inter alia di- or polyfunctional reagents, for example polyvalent metal ions or polyquaternary amines, for surface postcrosslinking. Here too improved SFC and PUP (Performance Under Pressure) values are observed after surface postcrosslinking has been carried out. To determine the PUP (Performance Under Pressure) values, the absorption capacity of the highly swellable hydrogels of a certain particle size fraction is measured under a pressure of 0.7 psi. Hydrogels of the particle size fraction 400 to 470 μm were measured in the present case. Table 1 compares the physical properties of hydrogels before and after surface postcrosslinking. Hydrogels from Nalco (Nalco 1180, non-surface-crosslinked) were treated with 1,3-dioxolan-2-one in aqueous solution so that the amount of surface crosslinking agent added was 5% by weight, based on the starting polymer. This treatment raised the PUP from 8.7 g/g to 29.3 g/g, and the SFC from 0.073×10⁻⁷ cm³sec/g to 115×10⁻⁷ cm³sec/g. Doubling the amount of 1,3-dioxolan-2-one added resulted in further improvements in SFC, but also in slightly decreasing PUP values. The following comparative values were obtained under changed experimental conditions:

| 1,3-Dioxolan-2-one | | |
|---|---|---|
| % by weight, based on starting | PUP | SFC |
| polymer | g/g | ×10⁻⁷ cm³sec/g |
| 5.25 | 30.6 | 44 |
| 10 | 30.0 | 69 |

This patent additionally captures the relationships between SFC, PUP and the particle size distribution as further parameters. The samples are commercially available polymer material from Stockhausen. Table 3 shows the following results:

| | Particle size | PUP | SFC |
|---|---|---|---|
| Sample | μm | g/g | ×10⁻⁷ cm³sec/g |
| 4-6 | 180-250 | 27.2 | 90 |
| 4-5 | 250-355 | 26.9 | 166 |
| 4-4 | 355-500 | 26.4 | 252 |
| 4-3 | 500-710 | 25.7 | 355 |

This experimental series shows the increase in SFC with increasing particle size distribution, whereas the Performance Under Pressure decreases.

A further way of obtaining good transportation properties would accordingly be to shift the particle size spectrum to higher values.

It is an object of the present invention to provide highly swellable hydrogels possessing fast acquisition times coupled with desired transportation properties and high ultimate absorption capacity. The hydrophilic swellable polymers shall have an absorption profile notable for properties such as high permeability and absorption capacity and also a fast swell rate.

We have found that this object is achieved, surprisingly, by increasing the crosslink density on the surface of or inside hydrogel-forming polymers of certain particle size distributions.

Another alternative are acidic hydrogel-forming polymers of certain particle size distribution.

The hydrogel material of the invention is thus very useful for a multiplicity of applications, for example the use to absorb aqueous fluids, for example in the food sector, in medicine, in building construction, in the agricultural industry or in fire protection.

The present invention relates to novel hydrophilic swellable polymers and their use for absorbing aqueous fluids, for example in the food sector, in medicine, in building construction, in the agricultural industry or in fire protection.

The invention provides especially hydrogel-forming acidic and/or surface-postcrosslinked polymers capable of absorbing aqueous fluids, wherein at least 80% by weight, i.e., 81, 82, 83, 84, 85, 86, 87, 88, 89% by weight, preferably 90% by weight, i.e., 91, 92, 93, 94% by weight, particularly preferably 95% by weight, i.e., 95.5, 96, 96.5% by weight, especially 97% by weight, i.e., 97.5, 98, 98.5, 99, 99.5, 99.6, 99.7, 99.8, 99.9% by weight of the particles have a particle size of less than 250 μm.

Acidic polymers are to be understood as meaning polymers having a pH of not more than 5.9 i.e., for example, 5.8 5.7 or 5.6, preferably not more than 5.5, i.e., for example, 5.4 or 5.3, more preferably not more than 5.2, i.e., for example, 5.1 and especially not more than 5.0, i.e., for example, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0 or less. The preferred pH range is between 1 and 5.9, more preferably between 3 and 5.9 and especially between 4 and 5.

The above-indicated weight percentages are preferably combined with a particle size upper limit of 200 μm, particularly preferably 160 μm, very particularly preferably 110 μm, especially 80 μm.

Surface-postcrosslinked polymers are polymers having a higher degree of crosslinking at the surface than in the center of the particles (core-shell structure). The surface postcrosslinking is preferably not effected using polyvalent metal ions.

Hydrogel-forming surface-postcrosslinked polymers or acidic optionally polymers capable of absorbing aqueous fluids are polymers capable of absorbing a multiple, especially at least 5 times, preferably 10 times, their weight of distilled water.

Preference is given to such hydrogel-forming polymers capable of absorbing aqueous fluids that have a CRC of greater than 17, 18, 19 or 20 g/g, preferably greater than 21, 22, 23, 24 or 25 g/g, especially greater than 26, 27, 28, 29, 30, or 31 g/g, or an AUL 0.3 psi of greater than 25, 26, 27, 28, 29, 30, or 31 g/g, preferably greater than 32, 33, 34, 35, 36 or 37 g/g, especially 38, 39, 40, 41, 42, 43 or 44 g/g. Preference is given to polymers which satisfy both the CRC and the AUL criteria. The hydrogel-forming surface-postcrosslinked polymers capable of absorbing aqueous fluids may optionally be inertized, for example with white oil.

Hydrogel-forming polymers capable of absorbing aqueous fluids are hydrogel-forming polymers capable of absorbing aqueous fluids that have been surface postcrosslinked or that have not been surface postcrosslinked. The non-surface-postcrosslinked polymers can arise as intermediates in surface postcrosslinking, but may in some instances also be used directly in the various applications.

Preferred hydrogel-forming polymers (optionally surface-postcrosslinked and/or acidic) capable of absorbing aqueous fluids are characterized by at least 80% by weight, i.e., 81, 82, 83, 84, 85, 86, 87, 88, 89% by weight, preferably 90% by weight, i.e., 91, 92, 93, 94% by weight, particularly preferably 95% by weight, i.e., 95.5, 96, 96.5% by weight, especially 97% by weight, i.e., 97.5, 98, 98.5, 99, 99.5, 99.6, 99.7, 99.8, 99.9% by weight, of the particles having a particle size of less than 250 μm and not more than 1% by weight, i.e., 0.9, 0.8, 0.7, 0.6, 0.5, 0.4% by weight, preferably not more than 0.3% by weight, i.e., 0.25, 0.2, 0.15% by weight, especially not more than 0.1% by weight, i.e., 0.09, 0.08, 0.07, 0.06, 0.05, 0.04% by weight, of the particles having a particle size distribution of less than 10 μm.

The abbve-indicated weight percentages are preferably combined with the particle size upper limit of 200 μm, particularly preferably 160 μm, very particularly preferably 110 μm, especially 80 μm.

Of hydrogel-forming polymers (optionally surface-postcrosslinked and/or acidic) capable of absorbing aqueous fluids, preference is given to those which, subject to the particle size upper limit of 250 μm, are characterized by not less than 60% by weight, i.e., 61, 62, 63, 64, 65, 66, 67, 68, 69% by weight, preferably not less than 70% by weight, i.e., 71, 72, 73, 74, 75, 76, 77, 78, 79% by weight, especially not less than 80% by weight, i.e., 81, 82, 83, 84, 85, 86, 87, 88, 89, 90% by weight, of the particles having a particle size distribution of greater than 30 μm and of less than 200 μm.

Of hydrogel-forming polymers (optionally surface-postcrosslinked and/or acidic) capable of absorbing aqueous fluids, preference is given to those which, subject to the particle size upper limit of 200 μm, are characterized by not less than 60% by weight, i.e., 61, 62, 63, 64, 65, 66, 67, 68, 69% by weight, preferably not less than 70% by weight, i.e., 71, 72, 73, 74, 75, 76, 77, 78, 79% by weight, especially not less than 80% by weight, i.e., 81, 82, 83, 84, 85, 86, 87, 88., 89, 90% by weight, of the particles having a particle size distribution of greater than 40 μm and of less than 180 μm.

Of hydrogel-forming polymers (optionally surface-postcrosslinked and/or acidic) capable of absorbing aqueous fluids, preference is given to those which, subject to the particle size upper limit of 160 μm, are characterized by not less than 60% by weight, i.e., 61, 62, 63, 64, 65, 66, 67, 68, 69% by weight, preferably not less than 70% by weight, i.e., 71, 72, 73, 74, 75, 76, 77, 78, 79% by weight, especially not less than 80% by weight, i.e., 81, 82, 83, 84, 85, 86, 87, 88, 89, 90% by weight, of the particles having a particle size distribution of greater than 15 μm and of less than 125 μm.

Of hydrogel-forming polymers (optionally surface-postcrosslinked and/or acidic) capable of absorbing aqueous fluids, preference is given to those which, subject to the particle size upper limit of 110 μm, are characterized by not less than 60% by weight, i.e., 61, 62, 63, 64, 65, 66, 67, 68, 69% by weight, preferably not less than 70% by weight, i.e., 71, 72, 73, 74, 75, 76, 77, 78, 79% by weight, especially not less than 80% by weight, i.e., 81, 82, 83, 84, 85, 86, 87, 88, 89, 90% by weight, of the particles having a particle size distribution of greater than 15 μm and of less than 90 μm.

Of hydrogel-forming polymers (optionally surface-postcrosslinked and/or acidic) capable of absorbing aqueous fluids, preference is given to those which, subject to the particle size upper limit of 80 μm, are characterized by not less than 60% by weight, i.e., 61, 62, 63, 64, 65, 66, 67, 68, 69% by weight, preferably not less than 70% by weight, i.e., 71, 72, 73, 74, 75., 76, 77, 78, 79% by weight, especially not less than 80% by weight, i.e., 81, 82, 83, 84, 85, 86, 87, 88, 89, 90% by weight, of the particles having a particle size distribution of greater than 15 μm and of less than 65 μm.

In addition, the following sieve cuts are preferred for the particle size upper limits of 250 μm, 200 μm, 160 μm, 110 μm and 80 μm: sieving through a 325 mesh sieve resulting in not less than 80% by weight, i.e., 81, 82, 83, 84, 85, 86, 87, 88, 89% by weight, preferably 90% by weight, i.e., 91, 92, 93, 94% by weight, particularly preferably 95% by weight, i.e., 95.5, 96, 96.5% by weight, especially 97% by weight, i.e., 97.5, 98, 98.5, 99, 99.5, 99.6, 99.7, 99.8, 99.9% by weight of the particles possessing a particle size of greater than 44 μm.

In addition, the following sieve cuts are preferred for the particle size upper limits of 250 μm, 200 μm, 160 μm and 110 μm: sieving through a 230 mesh-sieve-resulting in not less than 80% by weight, i.e., 81, 82, 83, 84, 85, 86, 87, 88, 89% by weight, preferably 90% by weight, i.e., 91, 92, 93, 94% by weight, particularly preferably 95% by weight, i.e., 95.5, 96, 96.5% by weight, especially 97% by weight, i.e., 97.5, 98, 98.5, 99, 99.5, 99.6, 99.7, 99.8, 99.9% by weight of the particles possessing a particle size of greater than 62 μm.

In addition, the following sieve cuts are preferred for the particle size upper limits of 250 μm, 200 μm, 160 μm and 110 μm: sieving through a 200 mesh sieve resulting in not less than 80% by weight, i.e., 81, 82, 83, 84, 85, 86, 87, 88, 89% by weight, preferably 90% by weight, i.e., 91, 92, 93, 94% by weight, particularly preferably 95% by weight, i.e., 95.5, 96, 96.5% by weight, especially 97% by weight, i.e., 97.5, 98, 98.5, 99, 99.5, 99.6, 99.7, 99.8, 99.9% by weight of the particles possessing a particle size of greater than 74 μm.

In addition, the following sieve cuts are preferred for the particle size upper limits of 250 μm, 200 μm and 160 μm: sieving through a 140 mesh sieve resulting in not less than 80% by weight, i.e., 81, 82, 83, 84, 85, 86, 87, 88, 89% by weight, preferably 90% by weight, i.e., 91, 92, 93, 94% by weight, particularly preferably 95% by weight, i.e., 95.5, 96, 96.5% by weight, especially 97% by weight, i.e., 97.5, 98, 98.5, 99, 99.5, 99.6, 99.7, 99.8, 99.9% by weight of the particles possessing a particle size of greater than 105 μm.

In addition, the following sieve cuts are preferred for the particle size upper limits of 250 μm and 200 μm: sieving through a 100 mesh sieve resulting in not less than 80% by weight, i.e., 81, 82, 83, 84, 85, 86, 87, 88, 89% by weight, preferably 90% by weight, i.e., 91, 92, 93, 94% by weight, particularly preferably 95% by weight, i.e., 95.5, 96, 96.5% by weight, especially 97% by weight, i.e., 97.5, 98, 98.5, 99, 99.5, 99.6, 99.7, 99.8, 99.9% by weight of the particles possessing a particle size of greater than 149 μm.

Narrower and wider particle size distributions can likewise be obtained through appropriate sieves or other methods of separation.

The inventive hydrogel-forming polymers capable of absorbing aqueous fluids preferably comprise a Vortex Time of less than 25 s, i.e., 24, 23, 22, 21 s, more preferably less than 20 s, i.e, 19, 18, 17, 16 s, even more preferably less than 15 s, i.e., 15 14, 13, 12, 11 s, yet more preferably less than 10 s, i.e., 9, 8 s, and especially less than 7 s, i.e, 6 or 5 s.

The inventive hydrogel-forming polymers capable of absorbing aqueous fluids have in (deionized) water after 10 min preferably an AUL (0.014 psi) of at least 20 g/g, i.e., for example, 21, 22, 23, 24 g/g or more, more preferably at least 25 g/g, i.e., for example 26, 27, 28, 29, g/g or more, even more preferably at least 30 g/g or more, especially of at least 40 g/g, i.e., for example, 41, 42, 43, 44, 45, 46, 47, 48, 49, g/g or more, or even of at least 50 g/g, i.e., for example, 51, 52, 53, 54, 55, 56, 57, 58, 59, 60 g/g or more.

The inventive hydrogel-forming polymers capable of absorbing aqueous fluids have in 0.9% NaCl solution after 10 min preferably an AUL (0.014 psi) of at least 15 g/g, i.e., for example, 16, 17, 18, 19 g/g or more, more preferably at least 22 g/g, 23 g/g, 24 g/g or more, even more preferably at least 25 g/g or 26 g/g or more, or even of 27 g/g, 28, 29 or 30 g/g or more.

The inventive hydrogel-forming polymers aqueous fluids have in 0.9% NaCl solution rapid absorptions. Preferably they have a difference in AUL (0.014 psi) between 60 and 10 minutes of less than 5 g/g, preferably of less than 4 g/g, more preferably of less than 3 g/g, even more preferably of less than 2 g/g and especially of less than 1 g/g. Moreover, preference is given to such polymers whose ratio of AUL (0.014 psi) at 10 min to 60 minutes is not less than 0.7, for example 0.71, 0.72, 0.73, 0.74 or more. Preference is given to ratios of 0.75 or more, for example 0.76, 0.78, 0.80, 0.82, 0.84, 0.86, 0.88 or more, more particular preference to ratios of 0.9 or more, for example 0.91, 0.92, 0.93, 0.94 or more, especially ratios of 0.95 or more, for example 0.96, 0.97, 0.98, 0.99, 1.00 or more. Preference is in addition given to such polymers whose ratio of AUL (0.014 psi) at 10 minutes to CRC is not less than 0.7, for example 0.72, 0.74, 0.76, 0.78, 0.80, 0.82, 0.84, 0.86, 0.88, 0.90, 0.92, 0.94, 0.96, 0.98 or more. Preference is given to ratios of 1.0 or more, for example 1.02, 1.04, 1.06, 1.08, 1.10, 1.12, 1.14, .1.16, 1.18 or more, particular preference to ratios of 1.2, for example 1.22, 1.24, 1.26, 1.28, 1.30, 1.32, 1.34, 1.36, 1.38 or more, especially to ratios of 1.4 or more, for example 1.42, 1.44, 1.46, 1.48, 1.50, 1.52, 1.54, 1.56, 1.58, 1.60 or more.

The invention further provides for the preparation of hydrogel-forming polymers capable of absorbing aqueous fluids by the various particle size distributions of the invention being set following surface postcrosslinking, for example by sieving. Optionally, it is also possible for surface postcrosslinking to be preceded by setting a certain fraction of particle size distribution (sieving, grinding, etc.) and subsequently certain cuts of particle size distribution being prepared after surface postcrosslinking. Alternatively, inventive hydrogel-forming polymers capable of absorbing aqueous fluids are prepared with a set particle size in such a way that no setting is needed for the particle size distribution of the invention after surface postcrosslinking. This can be accomplished for example by strong grinding or/and presieving.

The inventive hydrogel-forming polymers capable of absorbing fluids are useful in the hygiene sector for producing absorbent articles such as for example infant or adult diapers, incontinence articles or sanitary napkins and also in all other sectors outside hygiene which are concerned with the temporary or durable binding of aqueous fluids. Further uses can be in the fields of storage, packaging, transportation, food sector, medicine, cosmetics, textiles, chemical process industry applications, building construction, installation, water treatment, waste treatment, water removal, cleaning, agricultural industry and fire protection.

The particular advantages of the particle size distributions according to the invention reside in:

a) The particles according to the invention are swellable with defined amounts of water, pore-formers of various sizes being preparable depending on the amount of water, which, owing to the narrow particle size distribution, likewise cover a narrow, defined size range.

It is particularly advantageous to use surface-postcrosslinked superabsorbents when applications under pressure are concerned. Acidic superabsorbents are particularly advantageous in applications where rapid absorption is needed and especially in the case of applications where saline aqueous solutions have to be absorbed as well.

b) The incorporation of solid particles having a narrow particle size distribution into various materials of construction, for example sealing materials, films or cable sheaths, offers the advantage that the self-sealing effect wanted in the presence of water leads to a very uniform and rapid expansion of the surface area, since first, owing to a narrow particle size distribution, the swelling performance of all the particles is virtually identical and, secondly, large particles swell to a substantially greater extent due to water uptake than small ones, so that a broad particle size distribution has a substantially worse sealing effect.

c) It is likewise very important to have a very narrow particle size distribution in coextrusion, since otherwise very nonuniform surfaces, e.g. film surfaces, would result.

d) To produce thin layers having a very uniform surface, for example in fire protection. Here too it is advantageous to have particles having a very narrow distribution. Processed with water into a gel, such a gel can be spreadcoated or sprayed and can be formulated in such a way that it adheres to vertical walls for example.

e) Any hydrophilicization of surfaces is likewise only achievable when the surface remains very uniform and homogeneous following the uptake of water by the SAP. This can only be achieved with a narrow particle size distribution. The same applies to the uptake of condensation. The water should be absorbed quickly; hydrogels of this invention are best for this. In fruit and vegetable packs, the surface (of the tray or film) will change the most uniformly the better the homogeneity of the SAP particle size distribution. Specifically with regard to the uptake of condensation, whether in packages or in the building sector, i.e., wherever small amounts of water per unit time have to be absorbed over a prolonged period (and at irregular intervals), the small particles will absorb water substantially faster owing to the rate of incipient swell.

Methods of Making

a) Monomers Used

Hydrogel-forming polymers are in particular copolymers of (co)polymerized hydrophilic monomers, graft (co)polymers of one or more hydrophilic monomers on a suitable grafting base, crosslinked cellulose or starch ethers, crosslinked carboxymethylcellulose, partially crosslinked polyalkylene oxide or natural products that swell in aqueous fluids, for example guar derivatives, alginates and carrageenans. Suitable grafting bases can be of natural or synthetic origin. Examples are starch, cellulose or cellulose derivatives and also other polysaccharides and oligosaccharides, polyvinyl alcohol, polyalkylene oxides, especially polyethylene oxides and polypropylene oxides, polyamines, polyamides and also hydrophilic polyesters. Suitable polyalkylene oxides have for example the formula

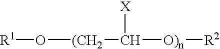

where

R¹ and R² are independently hydrogen, alkyl, alkenyl or acryl,

X is hydrogen or methyl and

is an integer from 1 to 10 000.

R¹ and R² are each preferably hydrogen, (C₁-C₄)-alkyl, (C₂-C₆)-alkenyl or phenyl.

Preferred hydrogel-forming polymers are crosslinked polymers having acid groups, which are predominantly in the form of their salts, generally alkali metal or ammonium salts. Such polymers. swell particularly strongly on contact with aqueous fluids to form gels.

Preference is given to polymers which are obtained by crosslinking polymerization or copolymerization of acid-functional monoethylenically unsaturated monomers or salts thereof. It is further possible to copolymerize these monomers without crosslinker and to crosslink them subsequently.

Examples of such monomers bearing acid groups are monoethylenically unsaturated C₃- to C₂₅-carboxylic acids or anhydrides such as acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. It is also possible to use monoethylenically unsaturated sulfonic or phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloyloxypropylsulfonic acid, 2-hydroxy-3-methacryloyloxypropylsulfonic acid, vinylphosphonic acid, allylphosphonic acid, styrenesulfonic acid and 2-acrylamido-2-methylpropanesulfonic acid. The monomers may be used alone or mixed.

Preferred monomers are acrylic acid, methacrylic acid, vinylsulfonic acid, acrylamidopropanesulfonic acid or mixtures thereof, for example mixtures of acrylic acid and methacrylic acid, mixtures of acrylic acid and acrylamidopropanesulfonic acid or mixtures of acrylic acid and vinylsulfonic acid.

To optimize properties, it can be sensible to use additional monoethylenically unsaturated compounds which do not bear an acid group but are copolymerizable with the monomers bearing acid groups. Such compounds include for example the amides and nitriles of monoethylenically unsaturated carboxylic acid, for example acrylamide, methacrylamide and N-vinylformamide, N-vinylacetamide,. N-methyl-N-vinylacetamide, acrylonitrile and methacrylonitrile. Examples of further suitable compounds are vinyl esters of saturated C₁- to C₄-carboxylic acids such as vinyl formate, vinyl acetate or vinyl propionate, alkyl vinyl ethers having at least 2 carbon atoms in the alkyl group, for example ethyl vinyl ether or butyl vinyl ether, esters of monoethylenically unsaturated C₃- to C₆-carboxylic acids, for example esters of monohydric C₁- to C18-alcohols and acrylic acid, methacrylic acid or maleic acid, monoesters of maleic acid, for example methyl hydrogen maleate, N-vinyllactams such as N-vinylpyrrolidone or N-vinylcaprolactam, acrylic and methacrylic esters of alkoxylated monohydric saturated alcohols, for example of alcohols having from 10 to 25 carbon atoms which have been reacted with from 2 to 200 mol of ethylene oxide and/or propylene oxide per mole of alcohol, and also monoacrylic esters and monomethacrylic esters of polyethylene glycol or polypropylene glycol, the molar masses (Mₙ) of the polyalkylene glycols being up to 2 000, for example. Further suitable monomers are styrene and alkyl-substituted styrenes such as ethylstyrene or tert-butylstyrene.

These monomers without acid groups may also be used in mixture with other monomers, for example mixtures of vinyl acetate and 2-hydroxyethyl acrylate in any proportion. These monomers without acid groups are added to the reaction mixture in amounts within the range from 0 to 50% by weight, preferably less than 20% by weight.

Preference is given to crosslinked polymers of monoethylenically unsaturated monomers which bear acid groups and which are optionally converted into their alkali metal or ammonium salts before or after polymerization and of 0-40% by weight, based on their total weight, of monoethylenically unsaturated monomers which do not bear acid groups.

Preference is given to crosslinked polymers of monoethylenically unsaturated C₃- to C₁₂-carboxylic acids and/or their alkali metal or ammonium salts. Preference is given in particular to crosslinked polyacrylic acids where 5-30 mol %, preferably 5-20 mol % and particularly preferably 5-10 mol % of the acid groups, based on the monomers containing acid groups, are present as alkali metal or ammonium salts.

Possible crosslinkers include compounds containing at least two ethylenically unsaturated double bonds. Examples of compounds of this type are N,N′-methylenebisacrylamide, polyethylene glycol diacrylates and polyethylene glycol dimethacrylates each derived from polyethylene glycols having a molecular weight of from 106 to 8 500, preferably from 400 to 2 000, trimethylolpropane triacrylate, trimethylolpropane trimethacrylate, ethylene glycol diacrylate, ethylene glycol dimethacrylate, propylene glycol diacrylate, propylene glycol dimethacrylate, butanediol diacrylate, butanediol dimethacrylate, hexanediol diacrylate, hexanediol dimethacrylate, allyl methacrylate, diacrylates and dimethacrylates of block copolymers of ethylene oxide and propylene oxide, polyhydric alcohols, such as glycerol or pentaerythritol, doubly or more highly esterified with acrylic acid or methacrylic acid, triallylamine, dialkyldiallylammonium halides such as dimethyldiallylammonium chloride and diethyldiallylammonium chloride, tetraallylethylenediamine, divinylbenzene, diallyl phthalate, polyethylene glycol divinyl ethers of polyethylene glycols having a molecular weight of from 106 to 4 000, trimethylolpropane diallyl ether, butanediol divinyl ether, pentaerythritol triallyl ether, reaction products of 1 mol of ethylene glycol diglycidyl ether or polyethylene glycol diglycidyl ether with 2 mol of pentaerythritol triallyl ether or allyl alcohol, and/or divinylethyleneurea. Preference is given to using water-soluble crosslinkers, for example N,N′-methylenebisacrylamide, polyethylene glycol diacrylates and polyethylene glycol dimethacrylates derived from addition products of from 2 to 400 mol of ethylene oxide with 1 mol of a diol or polyol, vinyl ethers of addition products of from 2 to 400 mol of ethylene oxide with 1 mol of a diol or polyol, ethylene glycol diacrylate, ethylene glycol dimethacrylate or triacrylates and trimethacrylates of addition products of from 6 to 20 mol of ethylene oxide with 1 mol of glycerol, pentaerythritol triallyl ether and/or divinylurea.

Possible crosslinkers also include compounds containing at least one polymerizable ethylenically unsaturated group and at least one further functional group. The functional group of these crosslinkers has to be capable of reacting with the functional groups, essentially the acid groups, of the monomers. Suitable functional groups include for example hydroxyl, amino, epoxy and aziridino groups. Useful are for example hydroxyalkyl esters of the abovementioned monoethylenically unsaturated carboxylic acids, e.g., 2-hydroxyethyl acrylate, hydroxypropyl acrylate, hydroxybutyl acrylate, hydroxyethyl methacrylate, hydroxypropyl methacrylate and hydroxybutyl methacrylate, allylpiperidinium bromide, N-vinylimidazoles, for example N-vinylimidazole, 1-vinyl-2-methylimidazole and N-vinylimidazolines such as N-vinylimidazoline, 1-vinyl-2-methylimidazoline, 1-vinyl-2-ethylimidazoline or 1-vinyl-2-propylimidazoline, which can be used in the form of the free bases, in quaternized form or as salt in the polymerization. It is also possible to use dialkylaminoalkyl acrylates and dialkylaminoalkyl methacrylates such as dimethylaminoethyl acrylate, dimethylaminoethyl methacrylate, diethylaminoethyl acrylate and diethylaminoethyl methacrylate. The basic esters are preferably used in quaternized form or as salt. It is also possible to use glycidyl(meth)acrylate, for example.

Useful crosslinkers further include compounds containing at least two functional groups capable of reacting with the functional groups, essentially the acid groups, of the monomers. Suitable functional groups were already mentioned above, i.e., hydroxyl, amino, epoxy, isocyanato, ester, amido and aziridino groups. Examples of such crosslinkers are ethylene glycol, diethylene glycol, triethylene glycol, tetraethylene glycol, polyethylene glycol, glycerol, polyglycerol, triethanolamine, propylene glycol, polypropylene glycol, block copolymers of ethylene oxide and propylene oxide, ethanolamine, sorbitan fatty acid esters, ethoxylated sorbitan fatty acid esters, trimethylolpropane, pentaerythritol, 1,3-butanediol, 1,4-butanediol, polyvinyl alcohol, sorbitol, starch, polyglycidyl ethers such as ethylene glycol diglycidyl ether, polyethylene glycol diglycidyl ether, glycerol diglycidyl ether, glycerol polyglycidyl ether, diglycerol polyglycidyl ether, polyglycerol polyglycidyl ether, sorbitol polyglycidyl ether, pentaerythritol polyglycidyl ether, propylene glycol diglycidyl ether and polypropylene glycol diglycidyl ether, polyaziridine compounds such as 2,2-bishydroxymethylbutanol tris[3-(1-aziridinyl)propionate], 1,6-hexamethylenediethyleneurea, diphenylmethanebis-4,4′-N,N′-diethyleneurea, haloepoxy compounds such as epichlorohydrin and α-methylepifluorohydrin, polyisocyanates such as 2,4-toluylene diisocyanate and hexamethylene diisocyanate, alkylene carbonates such as 1,3-dioxolan-2-one and 4-methyl-1,3-dioxolan-2-one, also bisoxazolines and oxazolidones, polyamidoamines and also their reaction products with epichlorohydrin, also polyquaternary amines such as condensation products of dimethylamine with epichlorohydrin, homo- and copolymers of diallyldimethylammonium chloride and also homo- and copolymers of dimethylaminoethyl(meth)acrylate which are optionally quaternized with, for example, methyl chloride.

Useful crosslinkers further include multivalent metal ions capable of forming ionic crosslinks. Examples of such crosslinkers are magnesium, calcium, barium and aluminum ions. These crosslinkers are used for example as hydroxides, carbonates or bicarbonates. Useful crosslinkers further include multifunctional bases likewise capable of forming ionic crosslinks, for example polyamines or their quaternized salts. Examples of polyamines are ethylenediamine, diethylenetriamine, triethylenetetramine, tetraethylenepentamine, pentaethylenehexamine and polyethyleneimines and also polyamines having molar masses in each case of up to 4 000 000.

The crosslinkers are present in the reaction mixture for example from 0.001 to 20% and preferably from 0.01 to 14% by weight. Ethoxylated trimethylolpropane triacrylate ETMPTA is a particularly preferred crosslinker.

b) Free Radical Polymerization

The polymerization is initiated in the generally customary manner, by means of an initiator. But the polymerization may also be initiated by electron beams acting on the polymerizable aqueous mixture. However, the polymerization may also be initiated in the absence of initiators of the abovementioned kind, by the action of high energy radiation in the presence of photoinitiators. Useful polymerization initiators include all compounds which decompose into free radicals under the polymerization conditions, for example peroxides, hydroperoxides, hydrogen peroxides, persulfates, azo compounds and redox catalysts. The use of water-soluble initiators is preferred. In some cases it is advantageous to use mixtures of different polymerization initiators, for example mixtures of hydrogen peroxide and sodium peroxodisulfate or potassium peroxodisulfate. Mixtures of hydrogen peroxide and sodium peroxodisulfate may be used in any proportion. Examples of suitable organic peroxides are acetylacetone peroxide, methyl ethyl ketone peroxide, tert-butyl hydroperoxide, cumene hydroperoxide, tert-amyl perpivalate, tert-butyl perpivalate, tert-butyl perneohexanoate, tert-butyl perisobutyrate, tert-butyl per-2-ethylhexanoate, tert-butyl perisononanoate, tert-butyl permaleate, tert-butyl perbenzoate, di(2-ethylhexyl)peroxydicarbonate, dicyclohexyl peroxydicarbonate, di(4-tert-butylcyclohexyl)peroxydicarbonate, dimyristyl peroxydicarbonate, diacetyl peroxydicarbonate, allyl peresters, cumyl peroxyneodecanoate, tert-butyl per-3,5,5-trimethylhexanoate, acetylcyclohexylsulfonyl peroxide, dilauryl peroxide, dibenzoyl peroxide and tert-amyl perneodecanoate. Particularly suitable polymerization initiators are water-soluble azo initiators, e.g., 2,2′-azobis(2-amidinopropane)dihydrochloride, 2,2′-azobis(N,N′-dimethylene)isobutyramidine dihydrochloride, 2-(carbamoylazo)isobutyronitrile, 2,2′-azobis[2-(2′-imidazolin-2-yl)propane]dihydrochloride and 4,4′-azobis(4-cyanovaleric acid). The polymerization initiators mentioned are used in customary amounts, for example in amounts of from 0.01 to 5%, preferably from 0.05 to 2.0%, by weight, based on the monomers to be polymerized.

Useful initiators also include redox catalysts. In redox catalysts, the oxidizing component is at least one of the above-specified per compounds and the reducing component is for example ascorbic acid, glucose, sorbose, ammonium or alkali metal bisulfite, sulfite, thiosulfate, hyposulfite, pyrosulfite or sulfide, or a metal salt, such as iron(II) ions or sodium hydroxymethylsulfoxylate. The reducing component in the redox catalyst is preferably ascorbic acid or sodium sulfite. Based on the amount of monomers used in the polymerization, from 3×10⁻⁶ to 1 mol % may be used for the reducing component of the redox catalyst system and from 0.001 to 5.0 mol % for the oxidizing component of the redox catalyst, for example.

When the polymerization is initiated using high energy radiation, the initiator used is customarily a photoinitiator. Photoinitiators include for example α-splitters, H-abstracting systems or else azides. Examples of such initiators are benzophenone derivatives such as Michler's ketone, phenanthrene derivatives, fluorene derivatives, anthraquinone derivatives, thioxanthone derivatives, coumarin derivatives, benzoin ethers and derivatives thereof, azo compounds such as the abovementioned free-radical formers, substituted hexaarylbisimidazoles or acylphosphine oxides. Examples of azides are: 2-(N,N-dimethylamino)ethyl 4-azidocinnamate, 2-(N,N-dimethylamino)ethyl 4-azidonaphthyl ketone, 2-(N,N-dimethylamino)ethyl 4-azidobenzoate, 5-azido-1-naphthyl 2′-(N,N-dimethylamino)ethyl sulfone, N-(4-sulfonylazidophenyl)maleimide, N-acetyl-4-sulfonylazidoaniline,-4-sulfonylazidoaniline, 4-azidoaniline, 4-azidophenacyl bromide, p-azidobenzoic acid, 2,6-bis(p-azidobenzylidene)cyclohexanone and 2,6-bis(p-azidobenzylidene)-4-methylcyclohexanone. Photoinitiators, if used, are customarily used in amounts of from 0.01 to 5% of the weight of the monomers to be polymerized.

The crosslinked polymers are preferably used in partially neutralized form. The degree of neutralization is generally in the range from 5 to 80%, preferably in the range from 5 to 60 mol %, more preferably in the range from 10 to 40 mol %, particularly preferably in the range from 20 to 30 mol %, based on the monomers containing acid groups. Useful neutralizing agents include alkali metal bases or ammonia/amines. Preference is given to the use of aqueous sodium hydroxide solution, aqueous potassium hydroxide solution or aqueous lithium hydroxide solution. However, neutralization may also be effected using sodium carbonate, sodium bicarbonate, potassium carbonate or potassium bicarbonate or other carbonates or bicarbonates or ammonia. Moreover primary, secondary and tertiary amines may be used.

Alternatively, the degree of neutralization can be set before, during or after the polymerization in all apparatuses suitable for this purpose. The neutralization can be effected for example directly in a kneader used for the polymerization.

Industrial processes useful for making these products include all processes which are customarily used to make superabsorbers, as described for example in Chapter 3 of “Modern Superabsorbent Polymer Technology”, F. L. Buchholz and A. T. Graham, Wiley-VCH, 1998.

Polymerization in aqueous solution is preferably conducted as a gel polymerization. It involves 10-70% strength by weight aqueous solutions of the monomers and optionally of a suitable grafting base being polymerized in the presence of a free-radical initiator by utilizing the Trommsdorff-Norrish effect.

The polymerization reaction may be carried out at from 0 to 150° C., preferably at from 10 to 100° C., not only at atmospheric-pressure but also at superatmospheric or reduced pressure. As is customary, the polymerization may also be conducted in a protective gas atmosphere, preferably under nitrogen.

By subsequently heating the polymer gels at from 50 to 130° C., preferably at from 70 to 100° C., for several hours, the performance characteristics of the polymers can be further improved.

c) Surface Postcrosslinking

A method for obtaining higher gel permeability is surface postcrosslinking, which provides higher gel strength to the hydrogel body in the swollen state. Gels having insufficient strength are deformable by pressure (as for example by denser packing in highly loaded systems), clog pores in the hydrogel absorbent and so prevent continued uptake of fluid. Since, for the reasons of decreasing absorption capacity values, an increased crosslink density in the starting polymer is out of the question, surface postcrosslinking is an elegant way to increase gel strength. Surface postcrosslinking increases the crosslink density in the shell of the hydrogel particles only, whereby the Absorbency Under.Load (AUL) of the base polymer thus generated is raised to a higher level. Whereas the absorption capacity decreases in the hydrogel shell, the core of the hydrogel particles has an improved absorption capacity (compared to the shell) owing to the presence of mobile polymer chains, so that sheath construction ensures improved fluid transmission. Depending on the field of use, it is accordingly possible to optimize absorption performance and gel strength through controlled adjustment of the degree of crosslinking in the base polymer and subsequent postcrosslinking and also by surface treatment of the polymer particles obtained.

Surface postcrosslinking may be carried out in a conventional manner using dried, ground and classified polymer particles of, for example, the size fraction less than 250 μm, 200 μm, 160 μm, 105 μm, preferably less than 63 μm.

It is likewise possible to feed the complete particle stream from grinding to the surface crosslinking stage and to effect the sieving to the desired particle size following surface crosslinking after the particles have dried.

In all cases, the surface postcrosslinking stage is optionally followed by (renewed) sieving to the desired particle size in order that any agglomerates which may be formed may be removed.

To effect surface postcrosslinking of the specified fractions of certain particle sizes, compounds capable of reacting with the functional groups of the polymers by crosslinking are applied to the surface of the hydrogel particles, preferably in the form of an aqueous solution. The aqueous solution may contain water-miscible organic solvents. Suitable solvents are alcohols such as methanol, ethanol, i-propanol or acetone.

The subsequent crosslinking reacts polymeric fines which have been prepared by the polymerization of the abovementioned monoethylenically unsaturated acids and optionally monoethylenically unsaturated monomers and which have a molecular weight of greater than 5 000, preferably greater than 50 000, with compounds which have at least two groups reactive toward acid groups. This reaction can take at room temperature or else at elevated temperatures up to 220° C.

Suitable postcrosslinkers include for example:

di- or polyglycidyl compounds such as diglycidyl phosphonates or ethylene glycol diglycidyl ether, bischlorohydrin ethers of polyalkylene glycols,

alkoxysilyl compounds,

polyaziridines, aziridine compounds based on polyethers or substituted hydrocarbons, for example bis-N-aziridinomethane,

polyamines or polyamidoamines and their reaction products with epichlorohydrin,

polyols such as ethylene glycol, 1,2-propanediol, 1,4-butanediol, glycerol, methyltriglycol, polyethylene glycols having an average molecular weight M_{w} of 200-10 000, di- and polyglycerol, pentaerythritol, sorbitol, the ethoxylates of these polyols and their esters with carboxylic acids or carbonic acid such as ethylene carbonate or propylene carbonate,

carbonic acid derivatives such as urea, thiourea, guanidine, dicyandiamide, 2-oxazolidinone and its derivatives, bisoxazoline, polyoxazolines, di- and polyisocyanates,

di- and poly-N-methylol compounds such as, for example, methylenebis(N-methylolmethacrylamide) or melamine-formaldehyde resins,

compounds having two or more blocked isocyanate groups such as, for example, trimethylhexamethylene diisocyanate blocked with 2,2,3,6-tetramethylpiperidin-4-one.

alkanolamines such as ethanolamine, diethanolamine, triethanolamine and the alkoxylated derivatives thereof.

If necessary, acidic catalysts may be added, for example p-toluenesulfonic acid, phosphoric acid, boric acid or ammonium dihydrogenphosphate.

Particularly suitable postcrosslinkers are di- or polyglycidyl compounds such as ethylene glycol diglycidyl ether, the reaction products of polyamidoamines with epichlorohydrin and 2-oxazolidinone and polyethylene glycol diacrylate.

The crosslinker solution is preferably applied to the hydrogels of defined particle size distribution by spraying with a solution of the crosslinker in conventional reaction mixers or mixing and drying equipment such as Patterson-Kelly mixers, DRAIS turbulence mixers, Lödige mixers, screw mixers, plate mixers, fluidized bed mixers and Schugi Mix. The spraying of the crosslinker solution may be followed by a heat treatment step, preferably in a downstream dryer, at from 80 to 230° C., preferably 80-190° C., particularly preferably at from 100 to 160° C., for from 5 minutes to 6 hours, preferably from 10 minutes to 2 hours, particularly preferably from 10 minutes to 1 hour, during which not only cracking products but also solvent fractions can be removed. But the drying may also take place in the mixer itself, by heating the jacket or by blowing in a preheated carrier gas.

In a particularly preferred embodiment of the invention, the hydrophilicity of the particle surface of the hydrogel-forming polymer is additionally modified by formation of complexes. The formation of complexes on the outer shell of the hydrogel particles is effected by spraying with solutions of divalent or more highly valent metal salt solutions, and the metal cations can react with the acid groups of the polymer to form complexes. Examples of divalent or more highly valent metal cations are Mg²⁺, Ca²⁺, Al³⁺, Sc³⁺, Ti⁴⁺, Mn²⁺, Fe^{2+/3+}, Co²⁺, Ni²⁺, Cu^{+/2+}, Zn²⁺, Y³⁺, Zr⁴⁺, Ag⁺, La³⁺, Ce⁴⁺, Hf⁴⁺, and Au^{+/3+}, preferred metal cations are Mg²⁺, Ca²⁺, Al³⁺, Ti⁴⁺, Zr⁴⁺ and La³+, and particularly preferred metal cations are Al³⁺, Ti⁴⁺ and Zr⁴⁺. The metal cations may be used not only alone but also mixed with each other. Of the metal cations mentioned, all metal salts are suitable that possess adequate solubility in the solvent to be used. Of particular suitability are metal salts with weakly complexing anions such as for example chloride, nitrate and sulfate. Useful solvents for the metal salts include water, alcohols, DMF, DMSO and also mixtures thereof. Particular preference is given to water and water/alcohol mixtures such as for example water-methanol or water-1,2-propanediol.

The spraying of the metal salt solution onto the particles of the hydrogel-forming polymer may be effected not only before but also after the surface postcrosslinking of the hydrogels of a certain particle size distribution. In a particularly preferred process, the spraying of the metal salt solution takes place in the same step as the spraying with the crosslinker solution, the two solutions being sprayed in succession or simultaneously via two nozzles or the crosslinker and metal salt solutions may be sprayed conjointly through a single nozzle.

Optionally, the hydrogel-forming particles may be further modified by admixture of finely divided inorganic solids, for example silica, alumina, titanium dioxide and iron(II) oxide, to further augment the effects of the surface aftertreatment. Particular preference is given to the admixture of hydrophilic silica or of alumina having an average primary particle size of from 4 to 50 nm and a specific surface area of 50-450 m²/g. The admixture of finely divided inorganic solids preferably takes place after the surface modification through crosslinking/complexing, but may also be carried out before or during these surface modifications. In general less than 5% by weight, preferably less than 1% by weight, in particular from 0.05 to 0.5% by weight, particularly preferably from 0.1 to 0.3% by weight, of solid are added.

Particular preference is given to the modification of the particle surface by the addition of oils, for example white oil. This dramatically reduces the dusting tendency of the hydrogels of a certain particle size distribution while minimally increasing the particle size. This type of modification is important in respect of product handling in particular, since dusting constitutes an enormous risk factor owing to the explosion hazard. In addition, the addition of white oil can prevent metering difficulties due to dusting.

Another useful way of suppressing dusting is the addition of glycerol and other di- and polyols, for example propylene glycol, ethylene glycol, polyethylene glycol and polypropylene glycol. The suppression of the tendency to dust is referred to as inertization.

A further modification option is to add surfactants. When these are in liquid form, they can likewise be used to control dusting owing to their ability to become optimally distributed on hydrophilic solid particles.

The modification of the the surface-postcrosslinked particles according to the invention may be effected following the surface postcrosslinking. But it is also possible to carry it out together with the surface postcrosslinking, for example via two nozzles, if the postcrosslinking is effected by spraying, or else by simply intermixing.

d) Properties of the Inventive Hydrogels of a Certain Particle Size Distribution

The inventive postcrosslinking hydrogel-forming polymer particles capable of absorbing aqueous fluids comprise an outer polymer shell of comparatively high crosslink density. This fact gives rise to an absorption profile which is notable for properties such as high gel strength and permeability coupled with high ultimate absorption capacity. Especially the Absorbency Under Load is raised to a higher level.

Increasing the crosslink density has the effect of increasing the gel strength of the individual particles, the consequence of which is that the absorption performance under confining pressure improves. By controlling the degree of crosslinking it is additionally possible to control certain values such as for example Absorbency Under Load or centrifuge retention. Surface postcrosslinking increases the permeability and optimizes the channelization of the aqueous fluids to be absorbed.

Similar effects can surprisingly also be provided by acidic polymers as per this invention and by polymers as per this invention whose particle sizes is reduced for very fine particles.

Advantages also result from the relatively large surface area of the multiplicity of small particles, which permits very short acquisition times and hence high incipient swell rates. The products of the invention may be aftertreated with white oil for example and constitute a powder without a tendency to dust despite the presence of fines. This permits safe handling of the product. It is thus very suitable for a multiplicity of different applications.

e) Use of the Inventive Hydrogels of Defined Particle Size Distribution

The present invention further provides for the use of the abovementioned hydrogel-forming polymers for absorbing aqueous fluids such as for example

hygiene articles,

storage, packaging, transportation (packaging material for water-sensitive articles, for example flower transportation, shock protection)

food sector (transportation of fish, fresh meat; absorption of water, blood in fresh fish/meat packs)

water treatment, waste treatment, water removal

cleaning

agricultural industry (irrigation, retention of meltwater and dew precipitates, composting additive)

The hydrogels of the particle size distribution according to the invention are suitable for the above applications; preferably they are used in combination with normal particle size distribution, and the advantages of the inventive hydrogels can be combined with those of the conventional hydrogels through an appropriate spatial configuration for example.

Particularly preferred applications for hydrogels of defined particle size distribution are:

medicine (wound plaster, water-absorbent material for burn dressings or for other weeping wounds, rapid dressings for injuries; rapid uptake of body fluid exudates for later analytical and diagnostic purposes), cosmetics, carrier material for pharmaceuticals and medicaments, rheumatic plaster, ultrasound gel, cooling gel, cosmetic thickener, sunscreen,

thickeners for oil/water or water/oil emulsions;

textile (gloves, sportswear, moisture regulation in textiles, shoe inserts, synthetic fabrics), hydrophilicization of hydrophobic surfaces; pore-forming

chemical process industry applications (catalyst-for organic reactions, immobilization of large functional molecules (enzymes), heat storage media, filtration aids, hydrophilic component in polymer laminates, dispersants, liquefiers)

building construction (sealing materials; systems or films that will self-seal in the presence of moisture; fine-pore formers in sintered building materials or ceramics; self-sealing insulation for water pipes or for underground pipes and tubes; sealing of building materials in the soil as a result of the SAP swelling in the moist soil with time delay and thus effecting a closure or seal; finishing of carpets and carpet floorings), installation, vibration-inhibiting medium, assistants in relation to tunneling in water-rich ground, assistants in relation to digging and boring in water-rich ground, cable sheathing

fire protection (spraying of SAP gel in the case of fires such as for example forest fires),

coextrusion agent in thermoplastic polymers; production of films and thermoplastic moldings capable of absorbing water (for example agricultural films capable of storing rain and dew water; SAP-containing films for keeping fresh fruit and vegetables which can packed in moist films to avoid fouling and wilting); SAP coextrudates, for example with polystyrene

carrier substance in active-ingredient formulations (drugs, crop protection)

agricultural industry: protection of forests against fungal and insect infestation, delayed release of active ingredients to plants)

The postcrosslinked hydrogel-forming particles of the invention are very useful as absorbents for water and aqueous fluids, can be used with advantage as water retainers in agricultural market gardening, as filtration aids and especially as an absorbent component in hygiene articles such as diapers, tampons or sanitary napkins.

Test Methods

a) Centrifuge Retention Capacity (CRC)

This method measures the free swellability of the hydrogel in a teabag. 0.2000±0.0050 g of a dried hydrogel are weighed into a teabag 60×85 mm in size which is subsequently sealed. The teabag is then placed for 30 minutes in an excess of 0.9% by weight sodium chloride solution (at least 0.83 1 of sodium chloride solution/1 g of polymer powder). The teabag is then centrifuged for 3 minutes at 250 g. The amount of liquid is determined by weighing back the centrifuged teabag.

b) Absorption Capacity (FSC Free Swell Capacity)

This method measures the free swellability of the hydrogel in a teabag. 0.2000±0.0050 g of a dried hydrogel are weighed into a teabag 60±85 mm in size which is subsequently sealed. The teabag is then placed for 30 minutes in an excess of 0.9% by weight sodium chloride solution (at least 0.83 1 of sodium chloride solution/1 g of polymer powder). The teabag is then suspended at one corner and allowed to drip for 10 minutes. The amount of liquid is determined by weighing back the teabag after the dripping has ended.

c) Absorbency Under Load (AUL) 0.3 psi

The measuring cell for determining AUL 0.3 psi is a Plexiglass cylinder 60 mm in internal diameter and 50 mm in height. Adhesively attached to its underside is a stainless steel sieve bottom having a mesh size of 36 μm. A Schleicher & Schmitt Schwarzband round filter (Ø 60 mm, pore size between 10-15 μm) is placed on the sieve bottom to prevent SAP particles having a particle size<36 μm falling through the meshes of the stainless steel sieve. The measuring cell further includes a plastic plate having a diameter of 59 mm and a weight which can be placed in the measuring cell together with the plastic plate. The plastic plate is loaded with the corresponding weight. AUL 0.3 psi is determined by determining the weight of the empty Plexiglass cylinder and of the plastic plate and recording it as W₀. 0.900±0.005 g of hydrogel-forming polymer is then weighed into the Plexiglass cylinder and distributed very uniformly over the round filter. The plastic plate is then carefully placed in the Plexiglass cylinder, the entire unit is weighed and the weight is recorded as Wₐ. The weight is then placed on the plastic plate in the Plexiglass cylinder. A ceramic filter plate 120 mm in diameter and 0 in porosity is then placed in the middle of a Petri dish 200 mm in diameter and 30 mm in height and sufficient 0.9% by weight sodium chloride solution is introduced for the surface of the liquid to be level with the filter plate surface without the surface of the filter plate being wetted. A round filter paper 90 mm in diameter and <20 μm in pore size (S&S 589 Schwarzband from Schleicher & Schüll) is subsequently placed on the ceramic plate. The Plexiglass cylinder containing hydrogel-forming polymer is then placed with plastic plate and weight on top of the filter paper and left there for 60 minutes. At the end of this period, the complete unit is removed from the Petri dish and subsequently the weight is removed from the Plexiglass cylinder. The Plexiglass cylinder containing swollen hydrogel is weighed together with the plastic plate, 0.4 g deducted as water absorption by the round filter and the weight recorded as W_{b}.

AUL was calculated by the following equation:

AUL 0.3 psi[g/g]=[W_{b}−Wₐ]/[Wₐ−Wₒ]

The weights are appropriately adapted in the case of AUL 0.2 psi, AUL 0.7 psi, etc. In the case of AUL (0.014 psi) without pressure, the measurement is carried out without weights, just with the plastic plate. For the time-dependent AUL values, the values are determined after certain times (2 min, 10 min, etc.). Instead of with 0.9% NaCl solution, the measurement can, for example, also be carried out in distilled water.

d) Vortex Time

50 ml of 0.9% by weight NaCl solution are measured into a 100 ml beaker. While the saline solution is being stirred with a rod-shaped magnetic stirrer (30 mm×8 mm) at 600 rpm, 2.00 g of hydrogel are poured in quickly in such a way that clumping is avoided. The time in seconds is taken for the vortex created by the stirring to close and for the surface of the saline solution to become flat.

e) Measurement of the Particle Size Distribution

The particle size distribution was determined by laser diffraction (instrument: Sympatec HELOS (H0173) RODOS).

f) pH Value Measurement

Carried out as per EDANA SAM-PHD-01-G protocol of February 99 bearing the reference pH 400.1-99. 0.5 g of superabsorbent 0.9% NaCl solution are measured with a pH electrode.

### EXAMPLES

### Example 1

A Werner & Pfleiderer laboratory kneader having a working capacity of 2 l is evacuated to 980 mbar absolute by means of a vacuum pump and a previously separately prepared monomer solution which has been cooled to about 25° C. and inertized by passing nitrogen into it is sucked into the kneader. The monomer solution has the following composition: 825.5 g of deionized water, 431 g of acrylic acid, 359 g of 50% NaOH, 0.86 g of polyethylene glycol 400 diacrylate (SARTOMER® 344 from CRAY VALLEY). To improve the inertization, the kneader is evacuated and subsequently refilled with nitrogen. This operation is repeated three times. A solution of 1.2 g of sodium persulfate (dissolved in 6.8 g of deionized water) is then sucked in, followed after a further 30 seconds by a further solution consisting of 0.024 g of ascorbic acid dissolved in 4.8 g of deionized water. After a nitrogen purge a preheated jacket heating circuit on bypass at 75° C. is switched over to the kneader jacket and the stirrer speed increased to 96 rpm. Following the onset of polymerization and the attainment of Tₘₐₓ, the jacket heating circuit is switched back to bypass, and the batch is supplementarily polymerized for 15 minutes without heating/cooling, subsequently cooled and discharged. The resultant gel particles are dried at 160° C. on wire mesh bottomed trays in a through air drying cabinet and then ground and sieved.

### Example 1a

The product thus obtained was sieved using a sieve with a mesh width of 105 μm. 1 200 g of the thus obtained product of particle size distribution <105 μm were sprayed with a homogeneous solution consisting of 20 g of water, 0.2 g of ethylene glycol diglycidyl ether and 0.66 g of sorbitan-monococoate in a powder mixing assembly (Lödige mixer) and transferred into a preheated second Lödige mixer. The heat treatment was carried out under constant conditions at a jacket temperature of 150° C. and a speed of 60 rpm for a period of 120 minutes. The mixer was emptied, and the product was cooled down to room temperature and again sieved off with a 105 μm sieve to remove agglomerates which may have formed. The performance data are shown in table 1.

### Example 1b

The postcrosslinking was carried out on the entire particle stream. 1 200 g of the resultant product of example 1 of particle size<850 μm were sprayed with a homogeneous solution consisting of 20 g of water, 0.2 g of ethylene glycol diglycidyl ether and 0.66 g of sorbitan monococoate in a powder mixing assembly (Lödige mixer) and transferred into a preheated second Lödige mixer. The heat treatment was carried out under constant conditions at a jacket temperature of 150° C. and a speed of 60 rpm for a period of 120 minutes. The mixer was emptied, and the product was cooled down to room temperature and again sieved off with a 105 μm sieve. The performance data are shown in table 1.

### Example 1c

The postcrosslinking was carried out on the entire particle stream. 1 200 g of the resultant product of example 1 of particle size <850 μm were sprayed with a homogeneous solution consisting of 20 g of water, 0.1 g of ethylene glycol diglycidyl ether and 0.33 g of sorbitan monococoate in a powder mixing assembly (Lödige mixer) and transferred into a preheated second Lödige mixer. The heat treatment was carried out under constant conditions at a jacket temperature of 150° C. and a speed of 60 rpm for a period of 120 minutes. The mixer was emptied, and the product was cooled down to room temperature and sieved off with a 105 μm sieve. The performance data are shown in table 1.

### Example 2

A Werner & Pfleiderer laboratory kneader having a working capacity of 2 l is evacuated to 980 mbar absolute by means of a vacuum pump and a previously separately prepared monomer solution which has been cooled to about 25° C. and inertized by passing nitrogen into it is sucked into the kneader. The monomer solution has the following composition: 825.5 g of deionized water, 431 g of acrylic acid, 359 g of 50% NaOH, 2.2 g of ethoxylated trimethylolpropane triacrylate ETMPTA (SARTOMER® 9035 from CRAY VALLEY). To improve the inertization, the kneader is evacuated and subsequently refilled with nitrogen. This operation is repeated three times. A solution of 1.2 g of sodium persulfate (dissolved in 6.8 g of deionized water) is then sucked in, followed after a further 30 seconds by a further solution consisting of 0.024 g of ascorbic acid dissolved in 4.8 g of deionized water. After a nitrogen purge a preheated jacket heating circuit on bypass at 75° C. is switched over to the kneader jacket and the stirrer speed increased to 96 rpm. Following the onset of polymerization and the attainment of Tₘₐₓ, the jacket heating circuit is switched back to bypass, and the batch is supplementarily polymerized for 15 minutes without heating/cooling, subsequently cooled and discharged. The resultant gel particles are dried at 160° C. on wire mesh bottomed trays in a through air drying cabinet and then ground and sieved.

### Example 2a

The product thus obtained was sieved using a sieve with a mesh size of 105 μm. 1 200 g of the thus obtained product of particle size distribution <105 μm were sprayed with a homogeneous solution consisting of 20 g of water, 0.2 g of ethylene glycol diglycidyl ether and 0.66 g of sorbitan monococoate in a powder mixing assembly (Lödige mixer) and transferred into a preheated second Lödige mixer. The heat treatment was carried out under constant conditions at a jacket temperature of 150° C. and a speed of 60 rpm for a period of 120 minutes. The mixer was emptied, and the product was cooled down to room temperature and sieved off with a 105 μm sieve to remove agglomerates which may have formed. The performance data are shown in table 1.

### Example 2b

The postcrosslinking was carried out on the entire particle stream. 1 200 g of the resultant product of example 2 of particle size <850 μm were sprayed with a homogeneous solution consisting of 20 g of water, 0.2 g of ethylene glycol diglycidyl ether and 0.66 g of sorbitan monococoate in a powder mixing assembly (Lödige mixer) and transferred into a preheated second Lödige mixer. The heat treatment was carried out under constant conditions at a jacket temperature of 150° C. and a speed of 60 rpm for a period of 120 minutes. The mixer was emptied, and the product was cooled down to room temperature and sieved off with a 105 μm sieve. The performance data are shown in table 1.

### Example 2c

The postcrosslinking was carried out on the entire particle stream. 1 200 g of the resultant product of example 2 of particle size <850 μm were sprayed with a homogeneous solution consisting of 20 g of water, 0.1 g of ethylene glycol diglycidyl ether and 0.33 g of sorbitan monococoate in a powder mixing assembly (Lödige mixer) and transferred into a preheated second Lödige mixer. The heat treatment was carried out under constant conditions at a jacket temperature of 150° C. and a speed of 60 rpm for a period of 120 minutes. The mixer was emptied, and the product was cooled down to room temperature and sieved off with a 105 μm sieve. The performance data are shown in table 1.

### Example 3

Carried out similarly to example 1.

### Example 3a

In contrast to the postcrosslinking under example 1, the heat treatment was in this case carried out for 70 minutes only. The postcrosslinking solution was made up directly before use. The two solutions (EGDGE and aluminum sulfate) were combined shortly upstream of the atomizer nozzle. The postcrosslinking solution for 1 200 g of powder (particle size distribution <105 μm) from inventive example 1 had the following composition: 17.58 g of water, 9.96 g of 1,2-propanediol, 1.2 g of ethylene glycol diglycidyl ether and 3.36 g of 26.8% aqueous aluminum sulfate solution. The performance data are shown in table 1.

### Example 4

Carried out similarly to example 2.

### Example 4a

In contrast to the postcrosslinking under example 2, the heat treatment was in this case carried out for 70 minutes only. The postcrosslinking solution was made up directly before use. The two solutions (EGDGE and aluminum sulfate) were combined shortly upstream of the atomizer nozzle. The postcrosslinking solution for 1 200 g of powder (particle size distribution <105 μm) from example 1 had the following composition: 17.58 g of water, 9.96 g of 1,2-propanediol, 1.2 g of ethylene glycol diglycidyl ether and 3.36 g of 26.8% aqueous aluminum sulfate solution. The performance data are shown in table 1.

### Example 5

Carried out similarly to example 1 but without postcrosslinking.

### Example 6

Carried out similarly to example 2 but without postcrosslinking. The comparative examples were tested on sieve fractions <105 μm

### Example 7

Carried out similarly to example 1. The postcrosslinking was effected according to method 1b. The polymer was not classified; the measurement was carried out on normal particle size distribution up to 850 μm.

### Example 8

Carried out similarly to example 1, except that 120 g of NaOH 50% were used. The polymer of example 8 has a pH of 4.44. The polymer is used as a base polymer, i.e., without further postcrosslinking. The performance data in 0.9% NaCl are discernible from table 2 and the performance data in water from table 3.

### Example 8a

The sieve fraction <63 μm corresponding to 96% by weight <110 μm from example 8 was used.

### Example 8b

The sieve fraction <100 μm corresponding to 95% by weight <200 μm from example 8 was used.

### Example 8c

The sieve fraction 63-100 μm corresponding to 96% by weight <160 μm from example 8 was used.

### Example 9

Example 9 is a highly swellable polymer which has not been surface postcrosslinked. The preparation of this polymer is precisely described in WO 00/22018 page 14 line 5-45. The performance data in 0.9% NaCl are discernible from table 2 and the performance data in water from table 3.

### Example 9a

The sieve fraction <63 μm corresponding to 96% by weight <110 μm from comparative example 8 was used.

### Example 9b

The sieve fraction <100 μm corresponding to 95% by weight <200 μm from comparative example 8 was used.

### Example 9c

The sieve fraction 63-100 μm corresponding to 96% by weight <160 μm from comparative example 8 was used.

### Example 10

The preparation of the base polymer is described in example 9.

Postcrosslinking was carried out on the entire particle stream. 1200 g of the resultant product of comparative example 8 of a particle size <850 μm were sprayed with a homogeneous solution consisting of 20 g of water, 0.2 g of ethylene glycol diglycidyl ether and 0.66 g of sorbitan monococoate in a powder mixing assembly (Lödige mixer) and transferred into a preheated second Lödige mixer. The heat treatment was carried out under constant conditions at a jacket temperature of 150° C. and a speed of 60 rpm for a period of 120 minutes. The mixer was emptied and the product cooled down to room temperature. The performance data in 0.9% NaCl are discernible from table 2 and the performance data in water from table 3.

### Example 10a

The sieve fraction <63 μm corresponding to 96% by weight <110 μm from comparative example 9 was used.

### Example 10b

The sieve fraction <100 μm corresponding to 95% by weight <200 from comparative example 9 was used.

### Example 10c

The sieve fraction 63-100 μm corresponding to 96% by weight <160 μm from comparative example 9 was used.

TABLE 1

| | Vortex | | AUL 0.3 | |
|---|---|---|---|---|
| | Time | CRC | psi | FSC |
| Example | S | g/g | g/g | g/g |
| 1a | 6 | 23.2 | 29.3 | 38.9 |
| 1b | 5 | 24.5 | 28.8 | 37.6 |
| 1c | 11 | 19.3 | 18.1 | 45.9 |
| 2a | 5 | 31.4 | 34.7 | 44.2 |
| 2b | 5 | 28.9 | 36.9 | 43.8 |
| 2c | 10 | 20.4 | 26.7 | 50.6 |
| 3 | 6 | 23.3 | 44.1 | 42.3 |
| 4 | 5 | 25.1 | 39.4 | 41.8 |
| 5 | 75 | 46.9 | 6.7 | 56.1 |
| 6 | 62 | 48.3 | 7.8 | 60.7 |
| 7 | 90 | 34.9 | 36.7 | 46.7 |

TABLE 2
Testing in 0.9% NaCl solution

| | | | | AUL | | | |
|---|---|---|---|---|---|---|---|
| | | AUL (1 h) | AUL (1 h) | (10 min) | | | |
| | Sieve | (0.7 psi) | (0.014 psi) | (0.014 psi) | FSC | | |
| Example | fraction | g/g | g/g | g/g | g/g | CRC g/g | pH |
| Example 8a) | <63 μm | 6.4 | 23.6 | 23.0 | 30.1 | 19.3 | 4.4 |
| Example 9a) | | 6.7 | 17.1 | 10.0 | 34.0 | 24.0 | 6.3 |
| Example 10a) | | 9.4 | 23.7 | 24.0 | 27.9 | 17.1 | 6.3 |
| Example 8b) | <100 μm | 6.4 | 24.6 | 24.5 | 29.7 | 19.2 | 4.4 |
| Example 9b) | | 6.8 | 29.0 | 18.9 | 46.0 | 34.3 | 6.3 |
| Example 10b) | | 9.0 | 24.0 | 24.9 | 26.9 | 18.1 | 6.4 |
| Example 8c) | 63-100 μm | 7.3 | 27.1 | 26.3 | 31.1 | 18.7 | 4.4 |
| Example 9c) | | 6.7 | 31.9 | 24.9 | 43.2 | 29.8 | 6.3 |
| Example 10c) | | 12.9 | 26.6 | 27.4 | 30.8 | 18.2 | 6.3 |

TABLE 3
Testing in water

| | | | AUL | | | |
|---|---|---|---|---|---|---|
| | AUL (1 h) | AUL (1 h) | (10 min) | | | |
| | (0.7 psi) | (0.014 psi) | (0.014 psi) | FSC | CRC | Vortex |
| Example | g/g | g/g | g/g | g/g | g/g | Time |
| Example 8a) | 9.0 | 74.3 | 43.7 | 193.2 | 125.8 | 12 s |
| Example 9a) | 9.1 | 32.7 | 20.6 | 221.4 | 170.4 | |
| Example | 11.4 | 48.8 | 29.5 | 90.5 | 61.3 | |
| 10a) | | | | | | |
| Example 8b) | 9.3 | 73.6 | 44.4 | 198.2 | 133.9 | 13 s |
| Example 9b) | 9.6 | 64.3 | 42.3 | 254.3 | 189.8 | |
| Example | 10.6 | 45.3 | 26.7 | 113.4 | 78.4 | |
| 10b) | | | | | | |
| Example 8c) | 10.0 | | | 197.9 | 136.1 | 9 s |
| Example 9c) | 9.3 | 82.8 | 50.1 | 239.4 | 170.4 | |
| Example | 15.8 | 92.1 | 57.4 | 131.4 | 79.6 | 10 s |
| 10c) | | | | | | |

1.-16. (Cancelled)

17. Hydrogel-forming polymer particles capable of absorbing aqueous fluids and having a pH of 5.9 or less, wherein 80% by weight of the particles have a particle size of less than 250 μm.

18. The polymer particles of claim 17 having a pH of 5.5 or less.

19. The polymer particles of claim 17 having a pH of 5.2 or less.

20. The polymer particles of claim 17 wherein 90% by weight of the particles have a particle size less than 250 μm.

21. The polymer particles of claim 17 wherein 95% by weight of the particles have a particle size of less than 250 μm.

22. The polymer particles of claim 17 wherein 97% by weight of the particles have a particle size of less than 250 μm.

23. The polymer particles of claim 17 wherein the particles are inertized.

24. The polymer particles of claim 17 wherein 60% by weight of the particles have a particle size distribution of greater than 30 μm and less than 200 μm.

25. The polymer particles of claim 17 wherein 70% by weight of the particles have a particle size distribution of greater than 30 μm and less than 200 μm.

26. The polymer particles of claim 17 wherein 80% by weight of the particles have a particle size of less than 160 μm.

27. The polymer particles of claim 17 wherein 90% by weight of the particles have a particle size of less than 160 μm.

28. The polymer particles of claim 17 wherein 80% by weight of the particles have a particle size of less than 110 μm.

29. The polymer particles of claim 17 wherein 90% by weight of the particles have a particle size of less than 110 μm.

30. The polymer particles of claim 17 wherein 80% by weight of the particles have a particle size of greater than 44 μm.

31. The polymer particles of claim 17 wherein 90% by weight of the particles have a particle size of greater than 44 μm.

32. The polymer particles of claim 17 having a 0.9% NaCl solution AUL (0.014 psi) after 10 minutes of at least 20 g/g.

33. The polymer particles of claim 17 having a ratio of AUL (0.014 psi) at 10 minutes to AUL (0.014 psi) at 60 minutes for 0.9% NaCl solution of 0.7 or more.

34. The polymer particles of claim 17 having a ratio of AUL (0.14 psi) at 10 minutes to CRC for 0.9% NaCl solution of 0.7 or more.

35. The polymer particles of claim 17 having a Vortex Time of less than 25 s.

36. Hydrogel-forming surface-post-crosslinked polymer particles capable of absorbing aqueous fluids, wherein 80% by weight of the particles have a particle size distribution of less than 250 μm.

37. Hydrogel-forming polymer particles capable of absorbing aqueous fluids wherein 80% by weight of the particles have a particle size of less than 250 μm, and not more than 1% by weight of the particles have a particle size of less than 10 μm.

38. The polymer particles of claim 37 wherein not more than 0.3% by weight of the particles have a particle size of less than 10 μm.

39. The polymer particles of claim 37 wherein not more than 0.1% by weight of the particles have a particle size of less than 10 μm.

40. A process for preparing polymer particles comprising providing a particle size distribution as set forth in claim 36 following surface postcrosslinking.

41. The process of claim 40 wherein the surface postcrosslinking is effected by spraying the particles and subsequent drying.

42. A process for preparing polymer particles comprising providing a particle size distribution as set forth in claim 23 following inertization.

43. A method of absorbing an aqueous fluid comprising contacting the fluid with the polymer particles of claim 17.
